(19) United States
(12) Patent Application Publication
(10) Pub. No.: US 2014/0276017 A1
(43) Pub. Date: Sep. 18, 2014

(54) SYSTEM FOR GUIDING WORKFLOW DURING A MEDICAL IMAGING PROCEDURE

(71) Applicant: VOLCANO CORPORATION, San Diego, CA (US)

(72) Inventor: Jason Sproul, San Diego, CA (US)

(73) Assignee: VOLCANO CORPORATION, San Diego, CA (US)

(21) Appl. No.: 14/213,062

(22) Filed: Mar. 14, 2014

Related U.S. Application Data
(60) Provisional application No. 61/781,600, filed on Mar. 14, 2013.

(51) Int. Cl. A61B 5/00; A61B 8/00; A61B 8/12

(52) U.S. Cl. CPC A61B 5/7475; A61B 8/12; A61B 5/0066; A61B 5/0084; A61B 8/54
USPC 600/425; 600/407; 600/467

(57) ABSTRACT

The invention includes patient interface modules that guide the workflow of an intravascular medical imaging procedure. In some instances a Patient Interface Modules (PIM) connected to the imaging catheter has one or more indicators that guide the workflow. Some modules may be adapted to connect to multiple imaging devices, or an imaging device and a pressure sensing device, or other treatment device.

### RELATED INVENTION

This application claims the benefit of, and priority to, U.S. Provisional application No. 61/781,600, filed Mar. 14, 2013, which is incorporated by reference in its entirety.

### FIELD OF THE INVENTION

The invention relates to patient interface modules that signal to an operator an order in which a medical imaging procedure should be carried out and methods of use thereof.

### BACKGROUND

Intravascular imaging techniques include ultrasound (IVUS) imaging and optical coherence tomography (OCT) imaging, among others. IVUS involves positioning an ultrasound transducer in a region of a vessel to be imaged, whereupon the transducer emits pulses of ultrasound energy into the vessel and surrounding tissue. A portion of the ultrasound energy is reflected off of the blood vessel wall and surrounding tissue back to the transducer. The reflected ultrasound energy (echo) impinges on the transducer, producing an electrical signal, which is used to form an image of the blood vessel. OCT involves directing an optical beam at a tissue from the end of a catheter and collecting the small amount of light that reflects from the tissue. Optical coherences between the source light and the reflected light can be used to determine tissue characteristics and to measure lumen size. In some instances, the imaging elements, mounted near the distal end of the catheter, are mechanically rotated during imaging, e.g., by rotating a drive cable coupled to a drive motor. The imaging elements may also be translated via mechanical devices external to the body.

Intravascular imaging provides details about cardiovascular health that cannot be obtained with non-invasive imaging such as CT and MRI. In particular the intravascular imaging can give information about the size and composition of tissues, such as thrombus, within the vasculature. The techniques are not limited to the vasculature, however, as the same techniques can be used generally to image luminal structures within a body.

While great strides have been made in simplifying intravascular imaging, it remains a delicate procedure that requires extensive training and a skilled practitioner. Mistakes can result in perforated arteries, or broken apparatus, which may block oxygen to tissues. In some instances, the imaging modalities described above are often used infrequently by an operator relative to other devices, and clinical staff may have difficulty recalling explicit procedures or the workflow between instruments. Additionally, when the procedures are done in an emergency setting, it can be difficult for the user to concentrate on the workflow because of other activity in the surrounding area.

### SUMMARY

The invention provides a Patient Interface Module (PIM) that provides a workflow guide to a user performing an intravascular imaging procedure. By following the workflow guide, users are reminded of a typical workflow and training is reinforced. The PIM is typically connected to an intravascular imaging catheter, such as an IVUS or OCT catheter. The PIM may be also connected to a separate control panel or interfaced to a computer. The PIM includes a plurality of buttons, which interactively signal a user to perform functions in a particular order. The PIM may include additional ports for receiving a medical imaging device configured for insertion into a body.

There are a variety of ways to indicate to an operator the next step in a workflow. In one exemplary embodiment, the signals are optical signals. For example, the control panel may highlight the button to be pushed by the operator. Typically, the module updates based on actions of the operator to signal the next button to be pressed. The module will generally include a connection to a computer. The connection can be wired or wireless.

The port of the module can be configured to receive any medical imaging device. In certain embodiments, the port is configured to receive a catheter. Any medical imaging catheter may be coupled to the port. Exemplary catheters include IVUS and/or OCT catheter. The module includes other features, such as drive and rotational motors so that systems of the invention can be used for pullback and rotational imaging procedures.

Another aspect of the invention provides methods for guiding an operator through a medical imaging procedure. Those methods involve providing a patient interface module. The module includes a control panel. The control panel includes a plurality of buttons. The control panels sends signals to an operator that provide an order in which a medical imaging procedure should be carried out based upon a sequence of the signals. The module also includes a port for receiving a medical imaging device configured for insertion into a body. Methods of the invention further involve signaling to the operator via the patient interface module an order in which a medical imaging procedure should be carried out.

### BRIEF DESCRIPTION OF THE DRAWINGS

FIG. 1 shows an exemplary catheter laboratory environment where a system of the invention can be used to guide workflow.

FIG. 2 shows an exemplary patient interface module (PIM) of the invention.

FIG. 3 shows a close-up of the keypad from FIG. 1.

FIG. 4 shows the relationship of the keypad on the PIM and the display on a computer.

FIG. 5 shows the connection of a PIM to a computer.

FIG. 6 shows a centralized workflow logic.

FIG. 7 shows a broadcasting workflow rule set.

FIG. 8 shows a distributed workflow logic.

### DETAILED DESCRIPTION

The invention generally relates to Patient Interface Modules (PIMs) that signal to a user an order in which a medical imaging procedure should be carried out and methods of use thereof. The invention provides systems and methods for coordinating operations during intravascular imaging. Any intravascular imaging system may be used in systems and methods of the invention. Systems and methods of the invention have application in intravascular imaging methodologies such as intravascular ultrasound (IVUS) and optical coherence tomography (OCT) among others that produce a three-dimensional image of a vessel. Some general aspects of PIMs are described in Kemp et al. (U.S. patent application number 2012/0165661), the content of which is incorporated by reference herein in its entirety.

FIG. 1 depicts an exemplary layout of an intravascular imaging system 101 as may be found, for example, in a catheter lab. An operator uses control station and navigational device 125 to operate catheter 112 via patient interface module (PIM) 105. At a distal tip of catheter 112 is imaging tip 114. Computer device 120 works with PIM 105 to coordinate imaging operations. Imaging operations proceed by using catheter 112 to image the patient's tissue. The image data is received by device 120 and interpreted to provide an image on monitor 103. System 101 is operable for use during diagnostic imaging of the peripheral and coronary vasculature of the patient. System 101 can be configured to automatically visualize boundary features, perform spectral analysis of vascular features, provide qualitative or quantitate blood flow data, or a combination thereof.

In some embodiments, operation of system 101 employs a sterile, single use intravascular ultrasound imaging catheter 112. Other embodiments may use an OCT imaging catheter. Catheter 112 is inserted into the coronary arteries and vessels of the peripheral vasculature under the guidance of angiographic system 107. System 101 may be integrated into existing and newly installed catheter laboratories (angiography suites.) The system configuration is flexible in order to fit into the existing catheter laboratory work flow and environment. For example, the system can include industry standard input/output interfaces for hardware such as navigation device 125, which can be a bedside mounted joystick. System 101 can include interfaces for one or more of an EKG system, exam room monitor, bedside rail mounted monitor, ceiling mounted exam room monitor, and server room computer hardware.

System 101 connects to catheter 112 via PIM 105, which may contain a type CF (intended for direct cardiac application) defibrillator proof isolation boundary. All other input/output interfaces within the patient environment may utilize both primary and secondary protective earth connections to limit enclosure leakage currents. The primary protective earth connection for controller 125 and control station 110 can be provided through the bedside rail mount. A secondary connection may be via a safety ground wire directly to the bedside protective earth system. Monitor 103 and an EKG interface can utilize the existing protective earth connections of the monitor and EKG system and a secondary protective earth connection from the bedside protective earth bus to the main chassis potential equalization post.

Computer device 120 can include a high performance dual Xeon based system using an operating system such as Windows XP professional or Windows 8. Computer device 120 may be configured to perform real time intravascular ultrasound imaging while simultaneously running a tissue classification algorithm referred to as virtual histology (VH). The application software can include a DICOM3 compliant interface, a work list client interface, interfaces for connection to angiographic systems, or a combination thereof. Computer device 120 may be located in a separate control room, the exam room, or in an equipment room and may be coupled to one or more of a custom control station, a second control station, a joystick controller, a PS2 keyboard with touchpad, a mouse, or any other computer control device.

Computer device 120 may generally include one or more USB or similar interfaces for connecting peripheral equipment. Available USB devices for connection include the custom control stations, the joystick, and a color printer. In some embodiments, control system includes one or more of a USB 2.0 high speed interface, a 50/100/1000 baseT Ethernet network interface, AC power input, PS2 jack, potential equalization post, 1 GigE Ethernet interface, microphone & line inputs, line output VGA Video, DVI video interface, PIM interface, ECG interface, other connections, or a combination thereof. As shown in FIG. 1, computer device 120 is generally linked to control station 110.

Control station 110 may be provided by any suitable device, such as a computer terminal (e.g., on a kiosk). In some embodiments, control system 110 is a purpose built device with a custom form factor (e.g., as shown in FIG. 12).

In certain embodiments, systems and methods of the invention include processing hardware configured to interact with more than one different three dimensional imaging system so that the tissue imaging devices and methods described here in can be alternatively used with OCT, IVUS, or other hardware.

Any target can be imaged by methods and systems of the invention including, for example, bodily tissue. In certain embodiments, systems and methods of the invention image within a lumen of tissue. Various lumen of biological structures may be imaged including, but not limited to, blood vessels, vasculature of the lymphatic and nervous systems, various structures of the gastrointestinal tract including lumen of the small intestine, large intestine, stomach, esophagus, colon, pancreatic duct, bile duct, hepatic duct, lumen of the reproductive tract including the vas deferens, vagina, uterus and fallopian tubes, structures of the urinary tract including urinary collecting ducts, renal tubules, ureter, and bladder, and structures of the head and neck and pulmonary system including sinuses, parotid, trachea, bronchi, and lungs.

In one embodiment, the imaging catheter is an IVUS catheter. IVUS catheters and processing of IVUS data are described for example in Yock, U.S. Pat. Nos. 4,794,931, 5,000,185, and 5,313,949; Sieben et al., U.S. Pat. Nos. 5,243,988, and 5,353,798; Crowley et al., U.S. Pat. No. 4,951,677; Pomeranz, U.S. Pat. No. 5,095,911, Griffith et al., U.S. Pat. No. 4,841,977, Maroney et al., U.S. Pat. No. 5,373,849, Born et al., U.S. Pat. No. 5,176,141, Lancee et al., U.S. Pat. No. 5,240,003, Lancee et al., U.S. Pat. No. 5,375,602, Gardineer et at., U.S. Pat. No. 5,373,845, Seward et al., Mayo Clinic Proceedings 71(7):629-635 (1996), Packer et al., Cardiostim Conference 833 (1994), “Ultrasound Cardioscopy,” Eur. J.C.P.E. 4(2):193 (June 1994), Eberle et al., U.S. Pat. No. 5,453,575, Eberle et al., U.S. Pat. No. 5,368,037, Eberle et at., U.S. Pat. No. 5,183,048, Eberle et al., U.S. Pat. No. 5,167,233, Eberle et at., U.S. Pat. No. 4,917,097, Eberle et at., U.S. Pat. No. 5,135,486, and other references well known in the art relating to intraluminal ultrasound devices and modalities.

In another embodiment, the invention provides a system for capturing a three dimensional image by OCT. Commercially available OCT systems are employed in diverse applications such as art conservation and diagnostic medicine, e.g., ophthalmology. OCT is also used in interventional cardiology, for example, to help diagnose coronary artery disease. OCT systems and methods are described in U.S. Pub. 2011/0152771; U.S. Pub. 2010/0220334; U.S. Pub. 2009/0043191; U.S. Pub. 2008/0291463; and U.S. Pub. 2008/0180683, the contents of each of which are hereby incorporated by reference in their entirety.

In OCT, a light source delivers a beam of light to an imaging device to image target tissue. Within the light source is an optical amplifier and a tunable filter that allows a user to select a wavelength of light to be amplified. Wavelengths commonly used in medical applications include near-infrared light, for example between about 800 nm and about 1700 nm.

Generally, there are two types of OCT systems, common beam path systems and differential beam path systems, which differ from each other based upon the optical layout of the systems. A common beam path system sends all produced light through a single optical fiber to generate a reference signal and a sample signal whereas a differential beam path system splits the produced light such that a portion of the light is directed to the sample and the other portion is directed to a reference surface. Common beam path interferometers are further described for example in U.S. Pat. No. 7,999,938; U.S. Pat. No. 7,995,210; and U.S. Pat. No. 7,787,127, the contents of each of which are incorporated by reference herein in its entirety.

In a differential beam path system, amplified light from a light source is input into an interferometer with a portion of light directed to a sample and the other portion directed to a reference surface. A distal end of an optical fiber is interfaced with a catheter for interrogation of the target tissue during a catheterization procedure. The reflected light from the tissue is recombined with the signal from the reference surface forming interference fringes (measured by a photovoltaic detector) allowing precise depth-resolved imaging of the target tissue on a micron scale. Exemplary differential beam path interferometers are Mach-Zehnder interferometers and Michelson interferometers. Differential beam path interferometers are further described for example in U.S. Pat. No. 7,783,337; U.S. Pat. No. 6,134,003; and U.S. Pat. No. 6,421,164, the contents of each of which are incorporated by reference herein in its entirety.

FIG. 2 shows an exemplary patient interface module (PIM) of the invention. The module may include more components than described or the module may include fewer or different components. FIG. 2 shows a control panel (PIM PCBA) that is coupled to a handheld keypad, and then an intravascular image catheter. FIG. 3 shows a close-up of the handheld keypad from FIG. 2. The control panel includes a plurality of buttons, as shown in FIG. 3. The buttons correspond to an order of events that occur to conduct a medical imaging procedure using a medical imaging device. In some embodiments, the buttons are backlit with a color such as red or green to indicate an order in which the steps should be performed.

The control panel sends signals to an operator that provide an order in which a medical imaging procedure should be carried out based upon a sequence of the signals. That is illustrated in FIG. 3, showing, for example, four buttons. Each button corresponds to an aspect of a workflow for conducting a medical imaging procedure. The operator is instructed by a signal from the control panel as to the button to push and when to push the button. As illustrated in FIG. 3, button 3 is indicated over buttons 1, 2, and 4. That signals to the operator that button 3 should be pushed to conduct that part of the medical imaging workflow. The PIM can be configured for any medical procedure by receiving data from a computer. FIG. 4 illustrates the relationship of the control panel to the computer.

There are a variety of ways to indicate to an operator the next step in a workflow. In the exemplary embodiment shown in FIGS. 3 and 4, the signals are optical signals. For example, the control panel may highlight the button to be pushed by the operator. Typically, the module updates based on actions of the operator to signal the next button to be pressed. Aspects of the invention are not limited to optical signals and any signaling method known in the art may be used with methods of the invention. The module will generally include a connection to a computer as shown in FIG. 5. The connection can be wired or wireless.

In the embodiment shown in FIG. 6, all control or visualization devices are synchronized with respect to the recommended or predicted next step, either by sharing a common logic entity, by maintaining separate but equivalent logic entities, or by broadcasting a rule set to each component which it then follows. FIG. 7 shows a broadcasting workflow rule set. Once rules have been broadcast to each component, events from each control device (e.g. GUI, PIM) are broadcast to all components. Said components then apply the rule set, potentially in combination with internal logic, to determine the state transitions appropriate to each given event. FIG. 8 shows a distributed workflow logic. In a purely distributed system, events are communicated amongst the components of the system. Each component then follows its own logic to determine the appropriate state transitions. This may in turn result in the emission of additional events. It should be apparent to a practitioner skilled in the art that the broadcast-rule and distributed workflows form two extremes of a continuum that encompasses many different possible embodiments.

In some embodiments different control and visualization devices may offer different recommended next steps based on the role of the user. This may correspond to varying sets of functionality and information provided to users with different roles such as a person delivering and monitoring therapy as contrasted with a person visualizing and assessing location, physiology, etc.

In some embodiments, the PIM predicts the next function required based on a recommended workflow (i.e., normative control) as it has been determined by the manufacturer. In other embodiments, the workflow may be determined by a specific user's typical workflow (i.e., individualized probabilistic control) allowing flexibility and inclusion of specific additional devices such as a pressure sensing guidewire. Other embodiments may develop a workflow through a learning algorithm that is reinforced with use (i.e., predictive control). Probabilistic or predictive techniques may include methods such as Bayesian models. Predictions may be based on one or more prior steps in the workflow, and may be weighted differently depending on the source of the input. For example, a clinical user operating a handheld device near the patient may have greater influence than a user operating a GUI in a remote control room.

In some embodiments, the PIM additionally includes ports for receiving medical imaging device(s) configured for insertion into a body. The port of the module can be configured to receive any medical imaging device. In certain embodiments, the port is configured to receive a catheter. Any medical imaging catheter may be coupled to the port. Exemplary catheters include IVUS and/or OCT catheter. Other devices such as sensing guidewires and treatment catheters may also be interfaced with the PIM. In embodiments using rotating imaging elements, the PIM may include other features, such as translational and rotational drive motors so that systems of the invention can be used for pullback and/or rotational imaging procedures.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A patient interface module, the module comprising:
a control panel comprising a plurality of buttons, wherein the control panels sends signals to an operator that provide an order in which a medical imaging procedure should be carried out based upon a sequence of the signals; and
a port for receiving a medical imaging device configured for insertion into a body.

2. The module according to claim 1, wherein the signals are optical signals.

3. The module according to claim 2, wherein the control panel highlights the button to be pushed by the operator.

4. The module according to claim 3, wherein the module updates based on actions of the operator to signal the next button to be pressed.

5. The module according to claim 1, further comprising a connection to a computer.

6. The module according to claim 5, wherein the connection is a wired connection.

7. The module according to claim 5, wherein the connection is a wireless connection.

8. The module according to claim 1, wherein the port is configured to receive a catheter.

9. The module according to claim 8, wherein the catheter is selected from the group consisting of an intravascular ultrasound catheter and an optical coherence tomography catheter.

10. The module according to claim 1, wherein module further comprises at least one motor.

11. A method for guiding an operator through a medical imaging procedure, the method comprising:
providing a patient interface module that comprising a control panel comprising a plurality of buttons, wherein the control panels sends signals to an operator that provide an order in which a medical imaging procedure should be carried out based upon a sequence of the signals; and a port for receiving a medical imaging device configured for insertion into a body; and
signaling to the operator via the patient interface module an order in which a medical imaging procedure should be carried out.

12. The method according to claim 11, wherein the signals are optical signals.

13. The method according to claim 12, wherein the control panel highlights the button to be pushed by the operator.

14. The method according to claim 13, wherein the module updates based on actions of the operator to signal the next button to be pressed.

15. The method according to claim 11, further comprising a connection to a computer.

16. The method according to claim 15, wherein the connection is a wired connection.

17. The method according to claim 15, wherein the connection is a wireless connection.

18. The method according to claim 11, wherein the port is configured to receive a catheter.

19. The method according to claim 18, wherein the catheter is selected from the group consisting of an intravascular ultrasound catheter and an optical coherence tomography catheter.

20. The method according to claim 11, wherein module further comprises at least one motor.
